(19) United States
(12) Patent Application Publication
(10) Pub. No.: US 2012/0184536 A1
(43) Pub. Date: Jul. 19, 2012

(54) COMPOSITIONS COMPRISING SPECIFIC UGT INHIBITORS AND METHODS OF USE THEREOF

(75) Inventor: Anna Radominska-Pandya, Little Rock, AR (US)

(73) Assignee: The Board of Trustees of the University of Arkansas, Little Rock, AR (US)

(21) Appl. No.: 13/406,177

(22) Filed: Feb. 27, 2012

Related U.S. Application Data
(63) Continuation-in-part of application No. PCT/US2010/047340, filed on Aug. 31, 2010.
(60) Provisional application No. 61/238,551, filed on Aug. 31, 2009.

(51) Int. Cl. A61K 31/506; C07D 489/02; C07D 405/04; C07D 487/04; A61K 31/5517; A61K 31/485

(52) U.S. Cl. 514/220; 514/274; 514/282; 544/312; 540/562; 546/44

(57) ABSTRACT

The present invention encompasses compositions comprising UGT inhibitors. Additionally, the invention encompasses methods of reducing the glucuronidation of a compound using a UGT inhibitor.

### CROSS REFERENCE TO RELATED APPLICATIONS

This application is a continuation-in-part of PCT Application PCT/US2010/047340, filed Aug. 31, 2010, which claims priority to U.S. provisional application No. 61/238,551, filed Aug. 31, 2009, each of which is hereby incorporated by reference in its entirety.

### GOVERNMENTAL RIGHTS

This invention was made with government support under grant number GM075893 awarded by the National Institutes of Health. The government has certain rights in the invention.

### BACKGROUND OF THE INVENTION

It is widely recognized that human UDP-glucuronosyltransferases (UGTs) are extensively involved in the overall metabolism and disposition of endo- and xenobiotics. UGTs catalyze the transfer of the glucuronosyl group from uridine 5′-diphospho-glucuronic acid (UDP-glucuronic acid) to substrate molecules that contain oxygen, nitrogen, sulfur or carboxyl functional groups Several human hepatic and extrahepatic UGTs have been identified as being involved in the glucuronidation of physiologically important substrates and drugs. Modulation of UGT activity by either inhibition or induction can alter the biotransformation of these substrates, active pharmaceutical ingredients and their rates of disposition. This can result in alteration of many pharmacological effects of these compounds and impact their pharmacokinetic properties. Currently, there are no specific UGT inhibitors available that could be used to slow undesired glucuronidation and turnover of specific drugs. Hence, there is a need in the art for compositions and methods for modulating glucuronidation.

### SUMMARY OF THE INVENTION

One aspect of the present invention encompasses a composition. The composition comprises a pharmaceutically active agent and a specific inhibitor of a UGT that glucuronidates the pharmaceutically active agent.

Another aspect of the present invention encompasses a method for decreasing the rate of glucuronidation of a compound in a subject. The method comprises administering the compound to the subject at substantially the same time as a UGT inhibitor specific for a UGT that glucuronidates the compound is administered.

Yet another aspect of the present invention encompasses a method for decreasing the clearance of AZT in a subject. The method comprises administering an inhibitor specific for UGT2B7 and AZT at substantially the same time as the AZT.

Still another aspect of the present invention encompasses a method for decreasing the clearance of morphine in a subject. The method comprises administering an inhibitor specific for UGT2B7 and morphine at substantially the same time as the morphine.

Other aspects and iterations of the invention are detailed below.

### REFERENCE TO COLOR FIGURES

The application file contains at least one photograph executed in color. Copies of this patent application publication with color photographs will be provided by the Office upon request and payment of the necessary fee

### BRIEF DESCRIPTION OF THE FIGURES

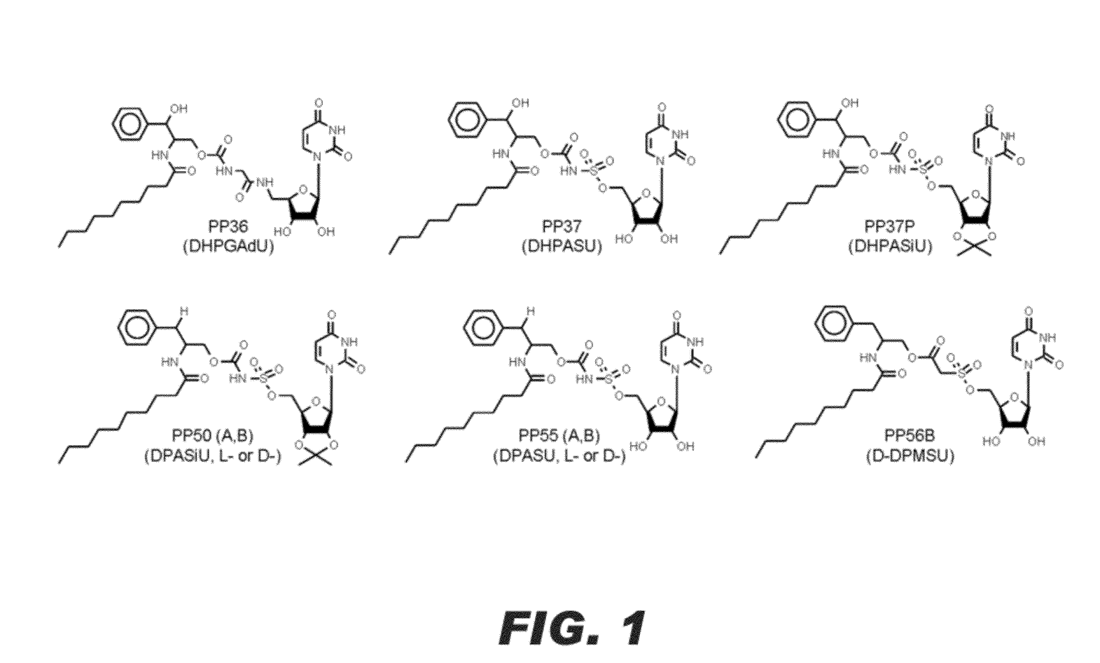

FIG. 1 depicts the structure of several PP-inhibitors.

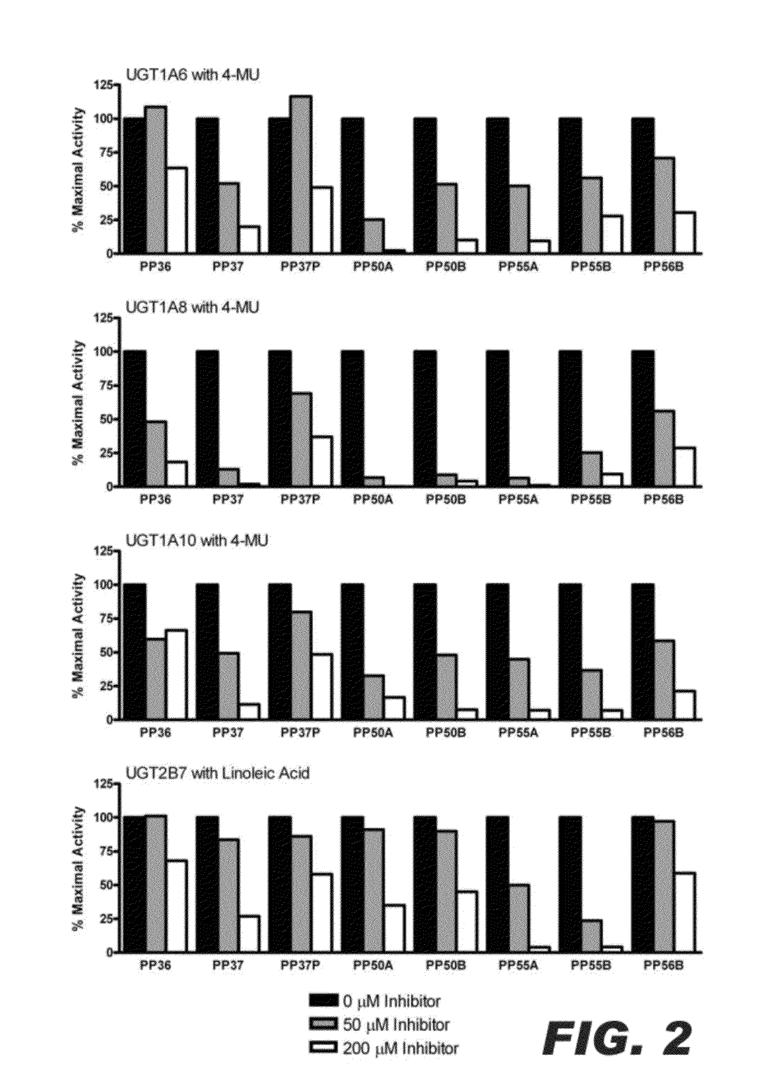

FIG. 2 depicts the effect of PP inhibitors on the glucuronidation by UGT1A6, 1A8, 1A10, and 2B7. Each enzyme was incubated with 250 μM of 4-MU or linoleic acid and 0 μM (black), 50 μM (grey), or 200 μM (white) of each inhibitor. Activities are normalized to percent maximal activity.

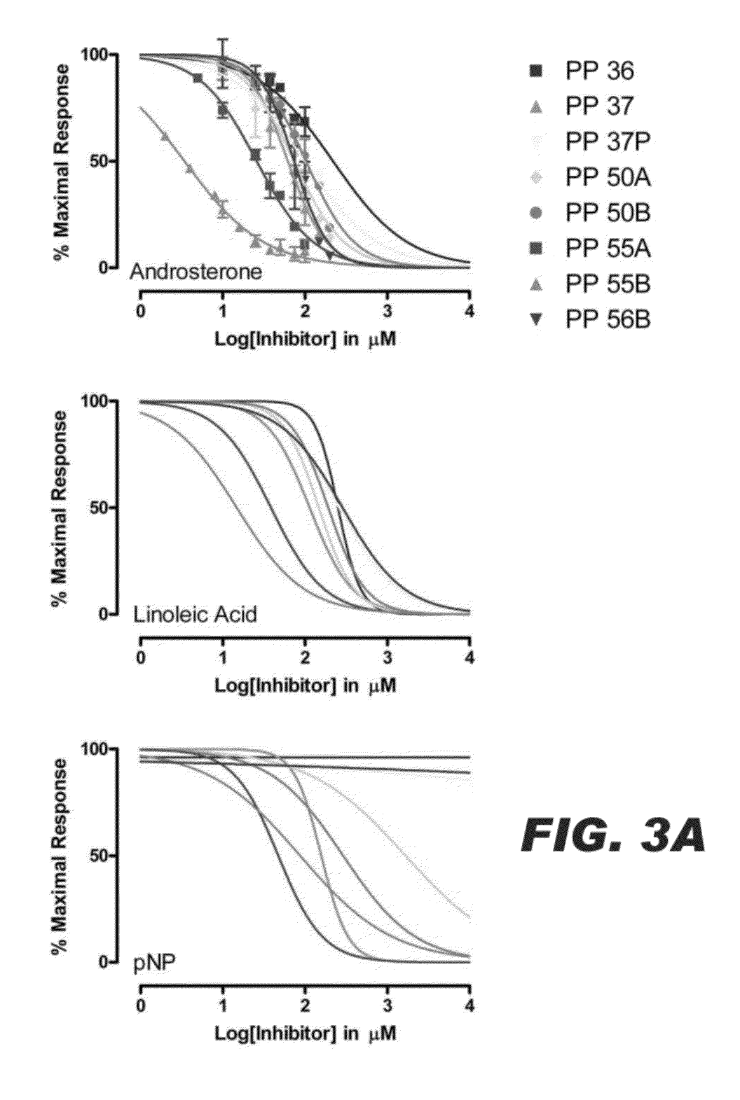

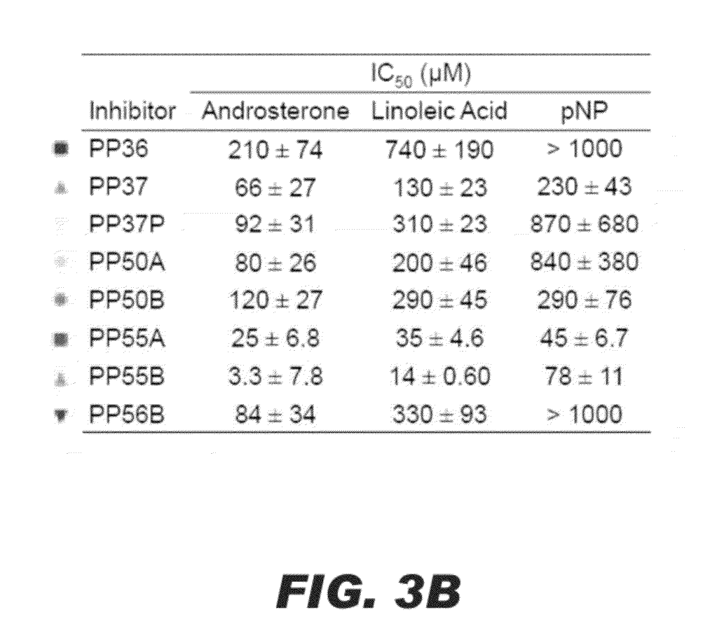

FIG. 3 depicts IC₅₀ calculations. UGT2B7 was incubated with a constant concentration of substrate and cosubstrate and increasing concentrations of each PPinhibitor. The IC₅₀ values were calculated and are shown in μM. The inhibition was assumed to be 100%.

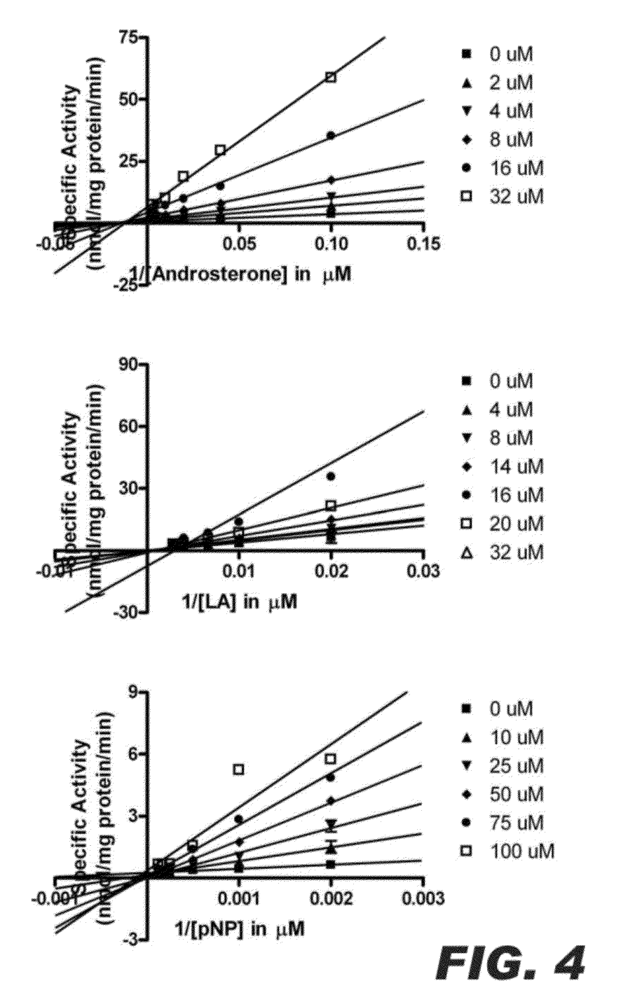

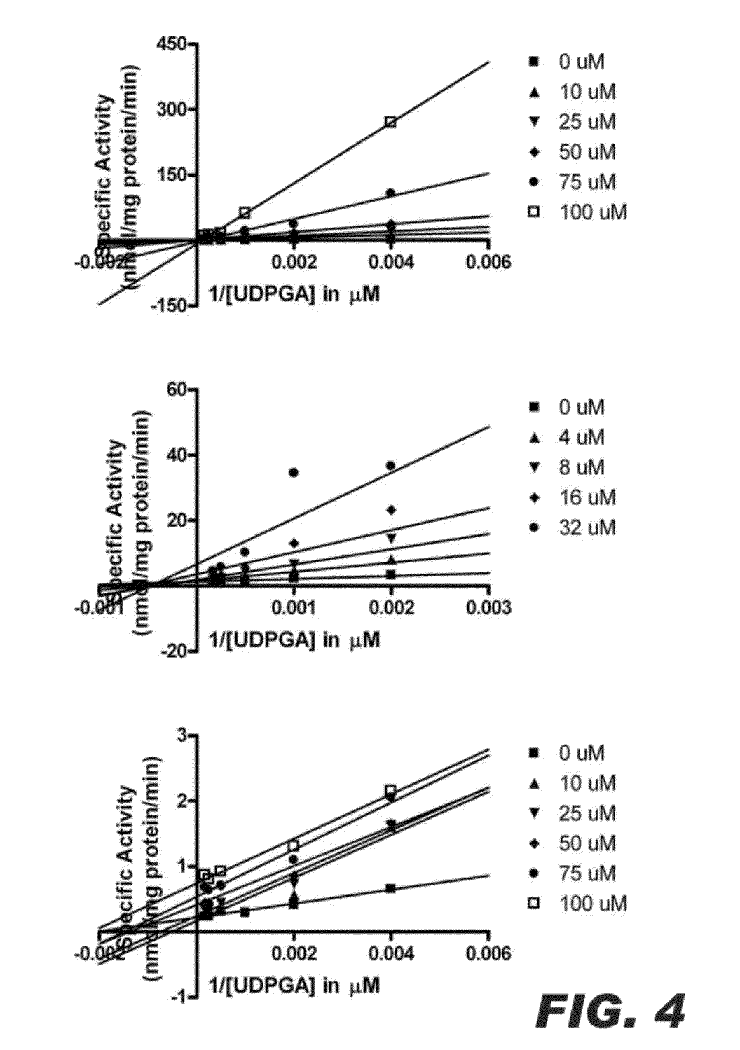

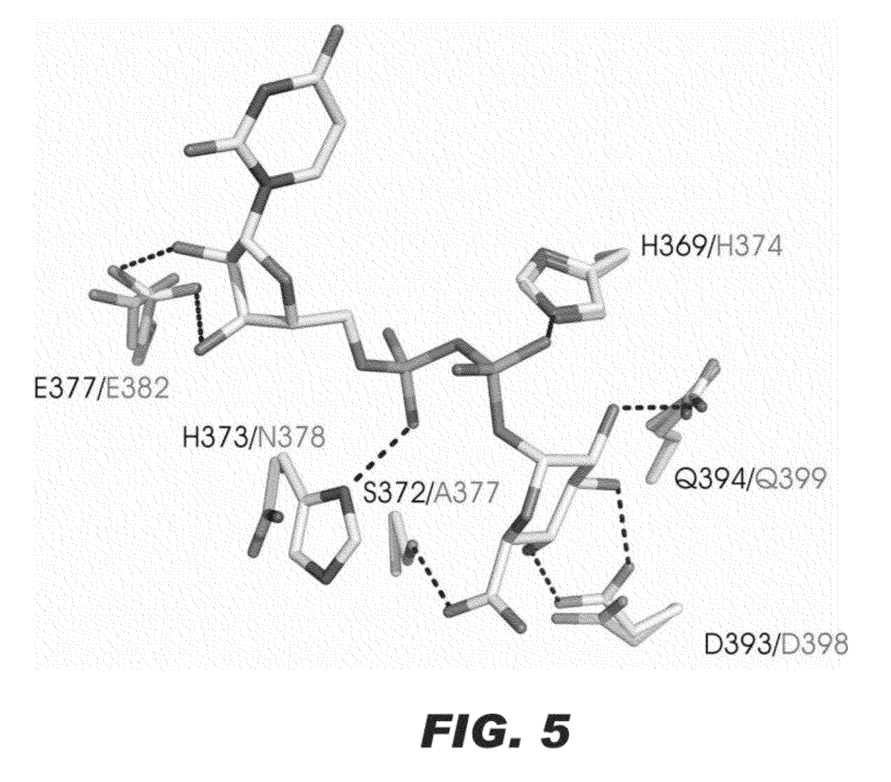

FIG. 4 depicts the determination of inhibition type. UGT1A10 was incubated with an increasing concentration of substrate at five fixed concentrations of inhibitor. First page of figures depicts substrate inhibition. Second page of figures depicts co-substrate inhibition. The data were graphed as 1/v against 1/[s].

FIG. 5 depicts a ball-and-stick representation of the model of the UDP-GA binding site in UGT1A10 (carbon, grey) and UGT2B7 (carbon, green). (In both models: nitrogen blue; oxygen red; phosphate orange). Dashed black lines represent possible hydrogen bonds. The bound co-substrate and the location of key residues in either UGT1A10 or UGT2B7 within hydrogen bonding distance. The residue types and numbering are indicated for both UGT1A10 (black) and UGT2B7 (green) are shown.

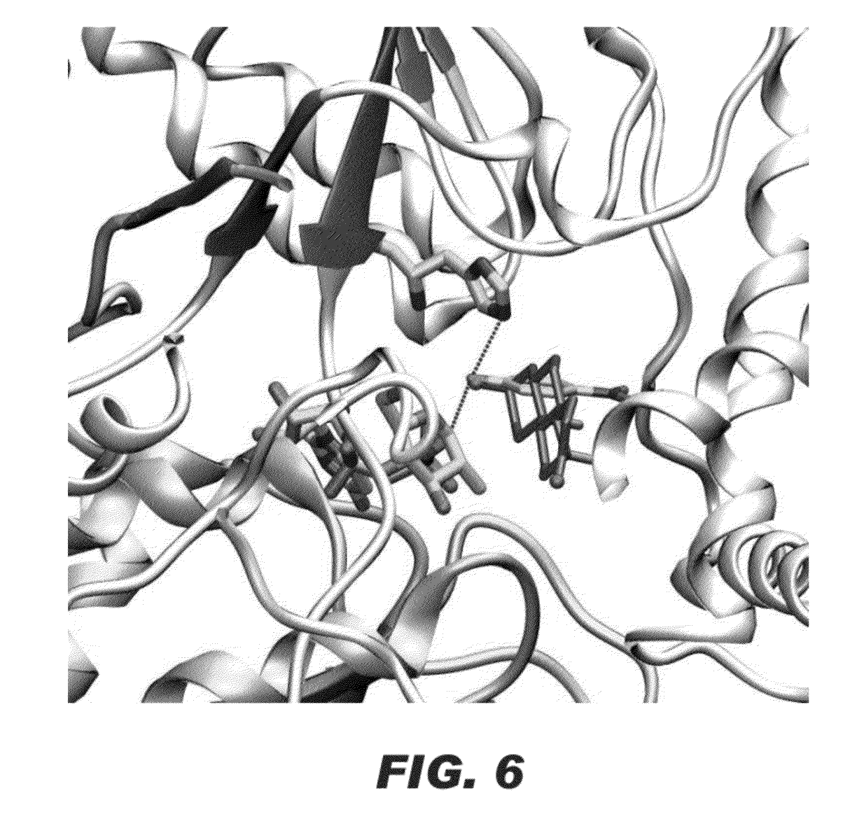

FIG. 6 depicts the active site of human UGT2B7 between the N-terminal (white) and C-terminal (green) domains. The beta-strands are shown as red arrows. Catalytically important residue His37 is shown in detail, are UDP-GlcUA and two substrates, pNP (green) and androsterone (blue). Substrates are aligned so that the reactive —OH from each is aligned between His37 and the reactive carbon of UDP-GlcUA.

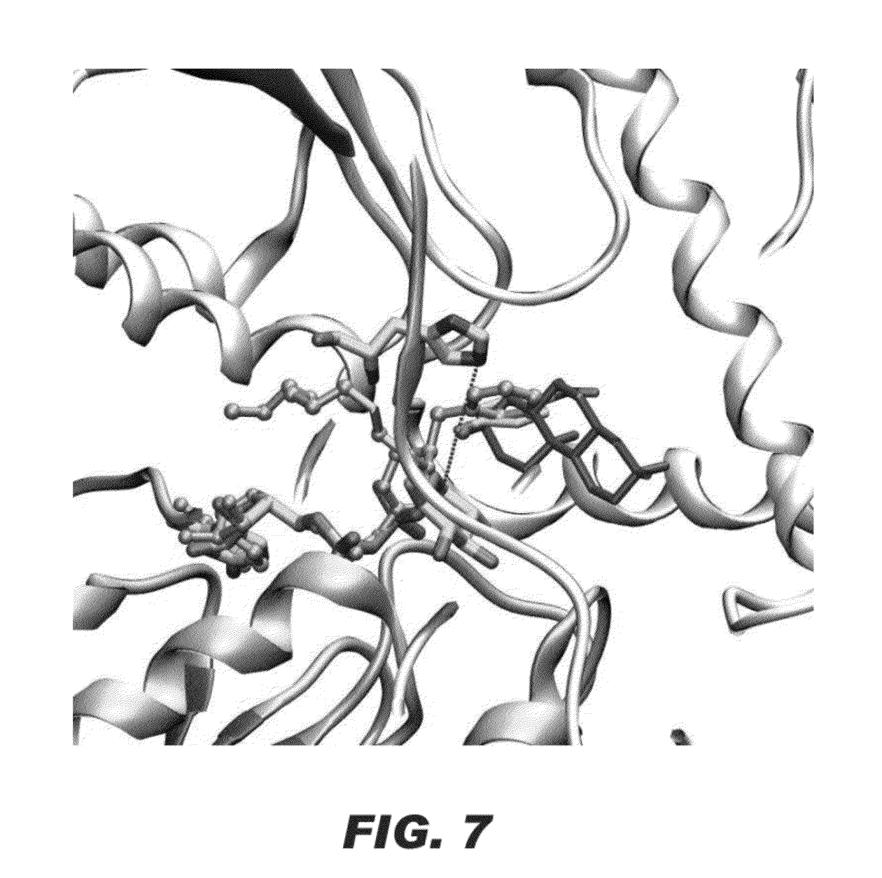

FIG. 7 depicts PP55B (orange) docked into the active site of human UGT2B7. PP55B is docked such that the uridine portion overlaps fully with the UDP-GlcUA. The aromatic ring of the inhibitor interferes with the binding positions of the two substrates, pNP (green) and androsterone (blue).

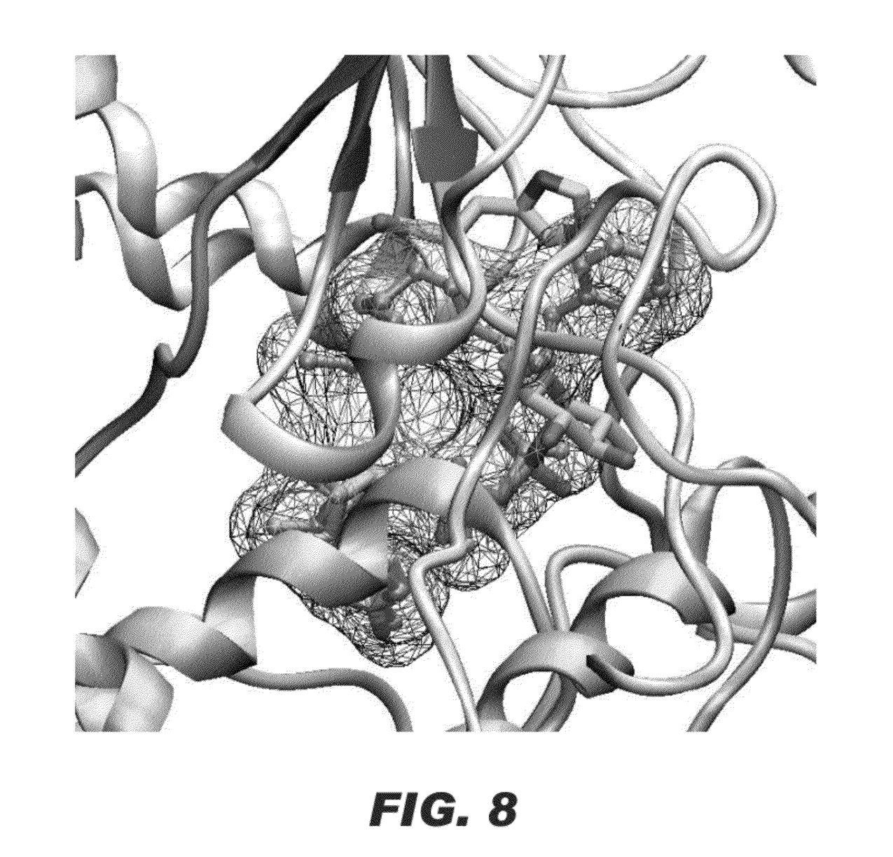

FIG. 8 depicts PP55B (orange) docked into the active site of human UGT2B7. PP55B is docked such that the uridine portion overlaps fully with the UDP-GlcUA. The mesh shows the molecular surface.

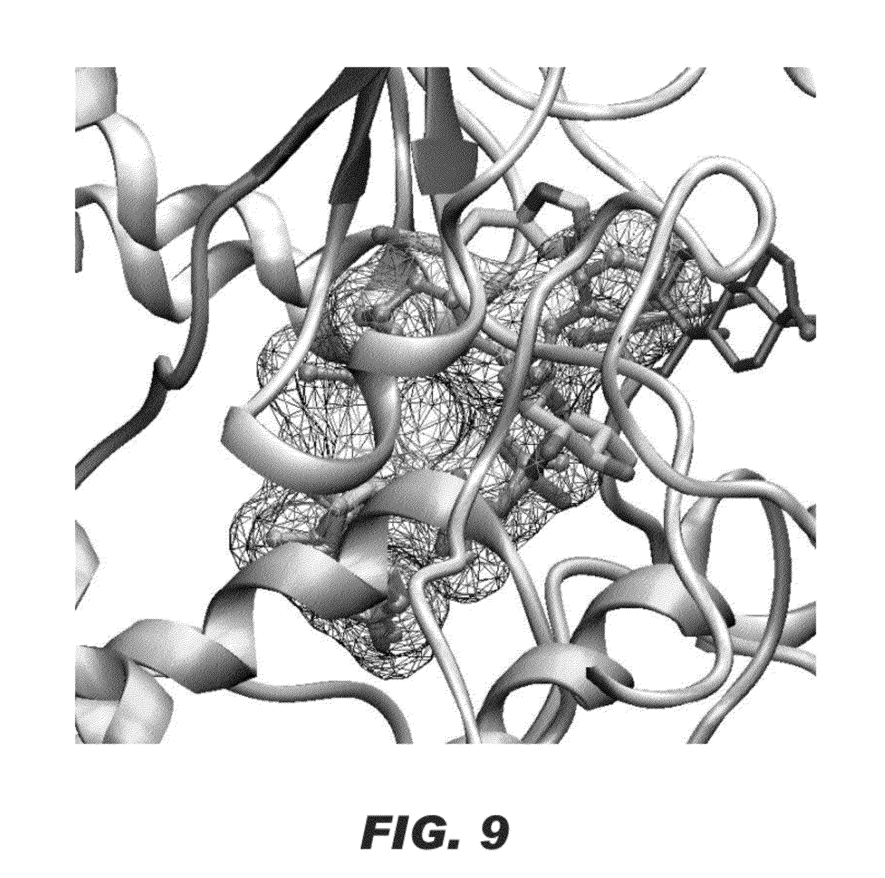

FIG. 9 depicts PP55B (orange) docked into the active site of human UGT2B7. PP55B is docked such that the uridine portion overlaps fully with the UDP-GlcUA. The mesh shows the molecular surface. PP55B clearly would not allow for the binding of either of the two substrates, pNP (green) and androsterone (blue).

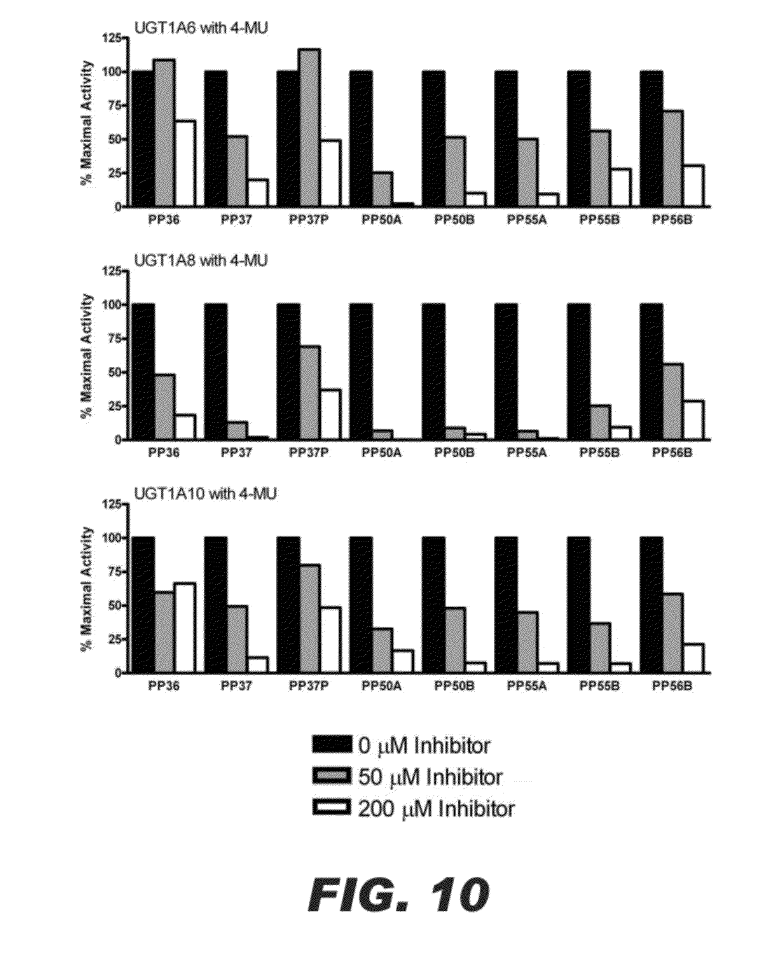

FIG. 10 depicts the effect of PP inhibitors on the glucuronidation of 4-MU by UGT1A6, 1A8, and 1A10. Each enzyme was incubated with 250 μM 4-MU and 0, 50, or 200 μM concentrations of each inhibitor. Activities are normalized to percent maximal activity.

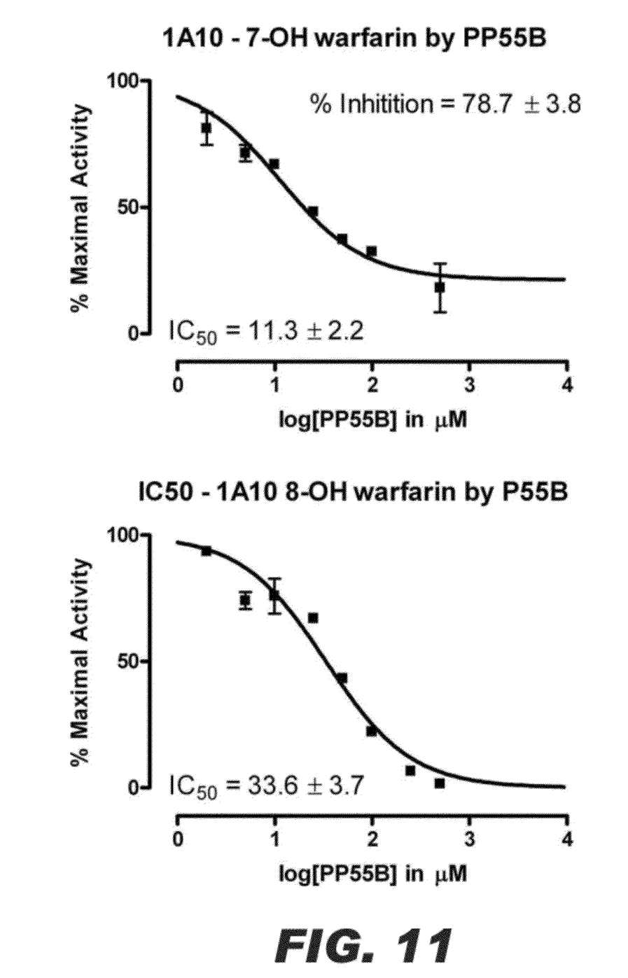

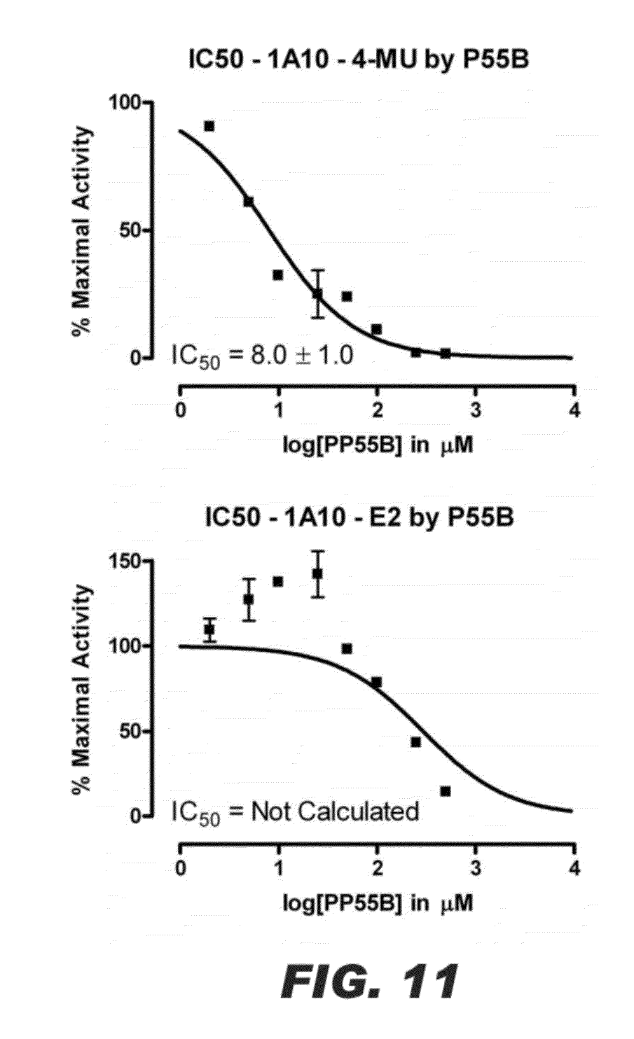

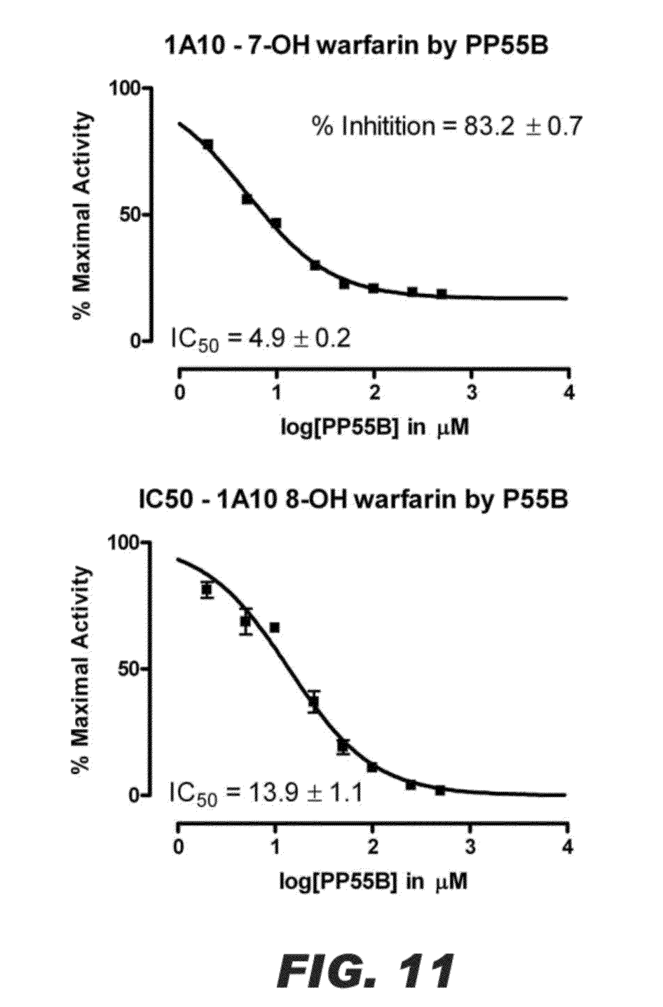

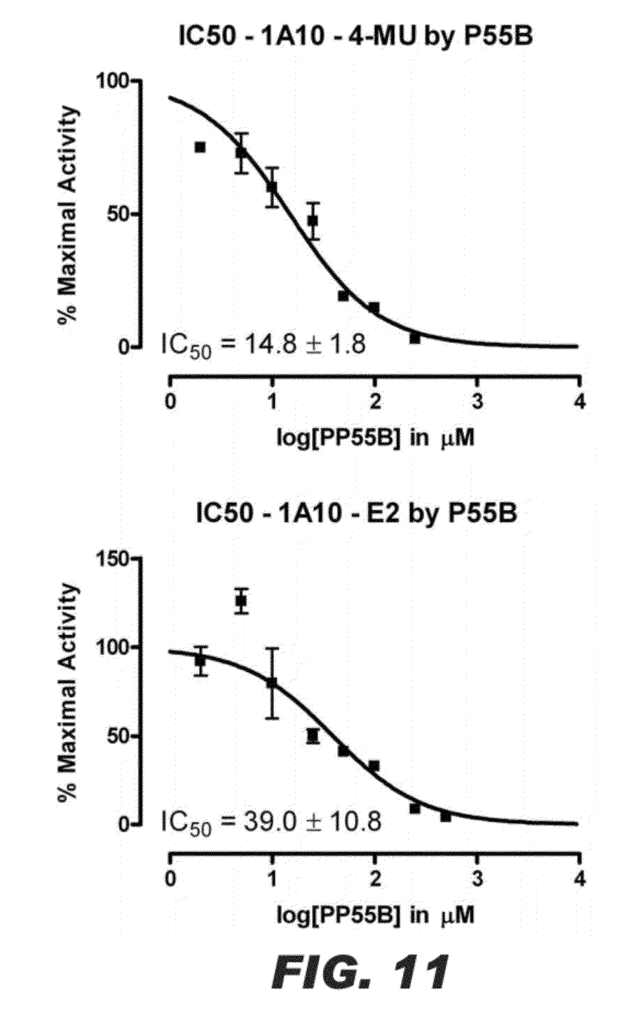

FIG. 11 depicts IC₅₀ calculations. UGT1A10 was incubated with a constant concentration of substrate (first two pages of figures) and co-substrate (second two pages of figures) and increasing concentrations of PP55B. The IC₅₀ values were calculated and are shown in μM. The inhibition was assumed to be 100% unless otherwise indicated.

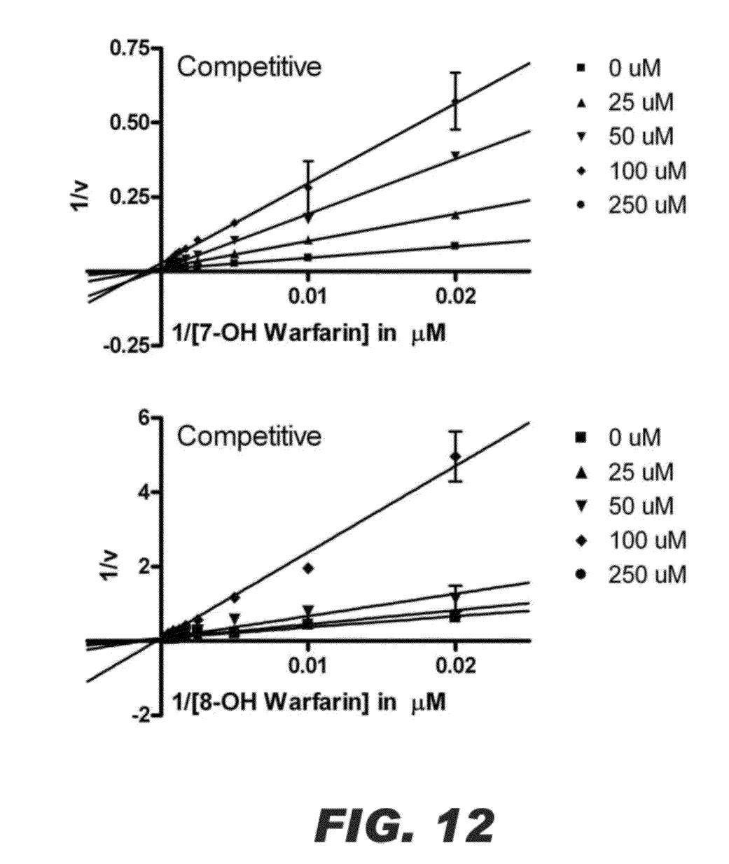

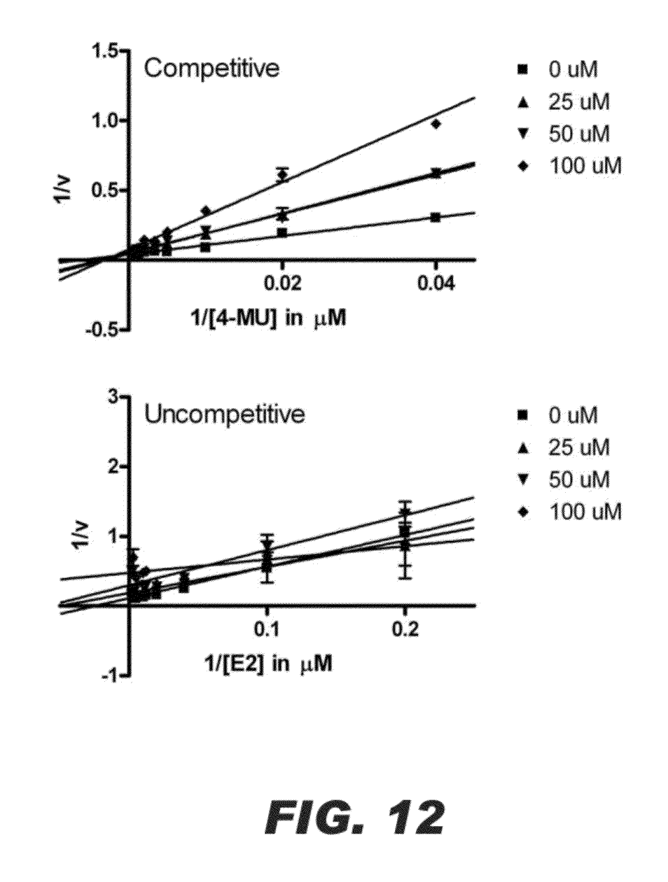

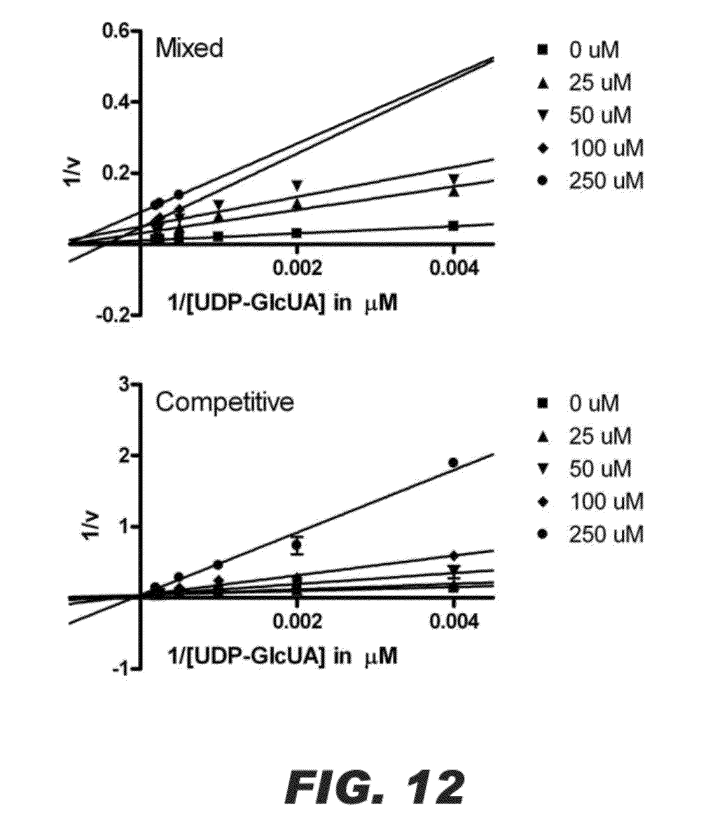

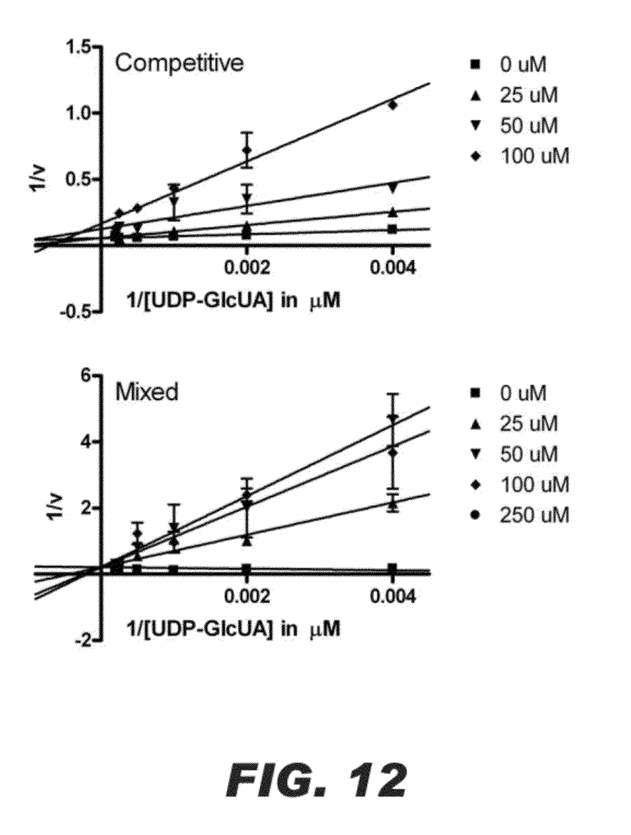

FIG. 12 depicts the determination of inhibition type. UGT1A10 was incubated with an increasing concentration of substrate at five fixed concentrations of inhibitor. First two pages of figures depict substrate inhibition. Second two pages of figures depict co-substrate inhibition. The data was graphed as 1/v against 1/[s].

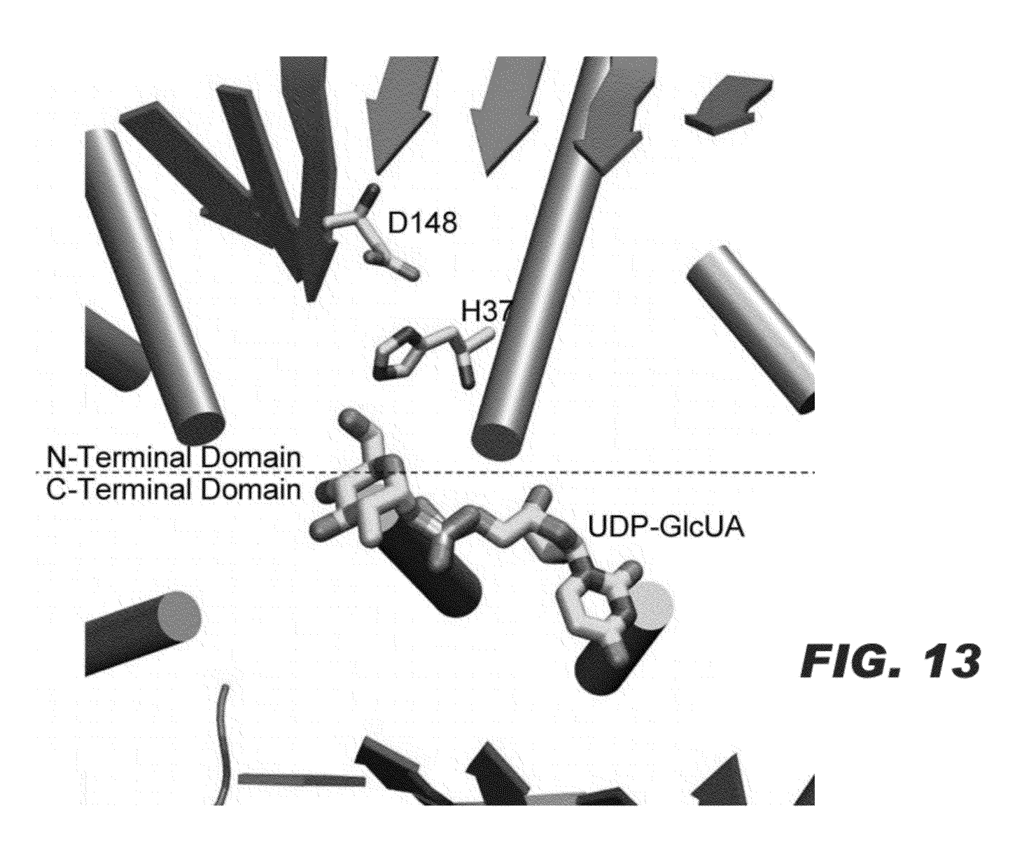

FIG. 13 depicts the active site of human UGT1A10 between N- and C-terminal domains. The helixes are shown as ice blue cylinders, and the beta-strands as red arrows. Catalytically important residues His37 and Asp148 that are shown in detail, as is also a substrate analog.

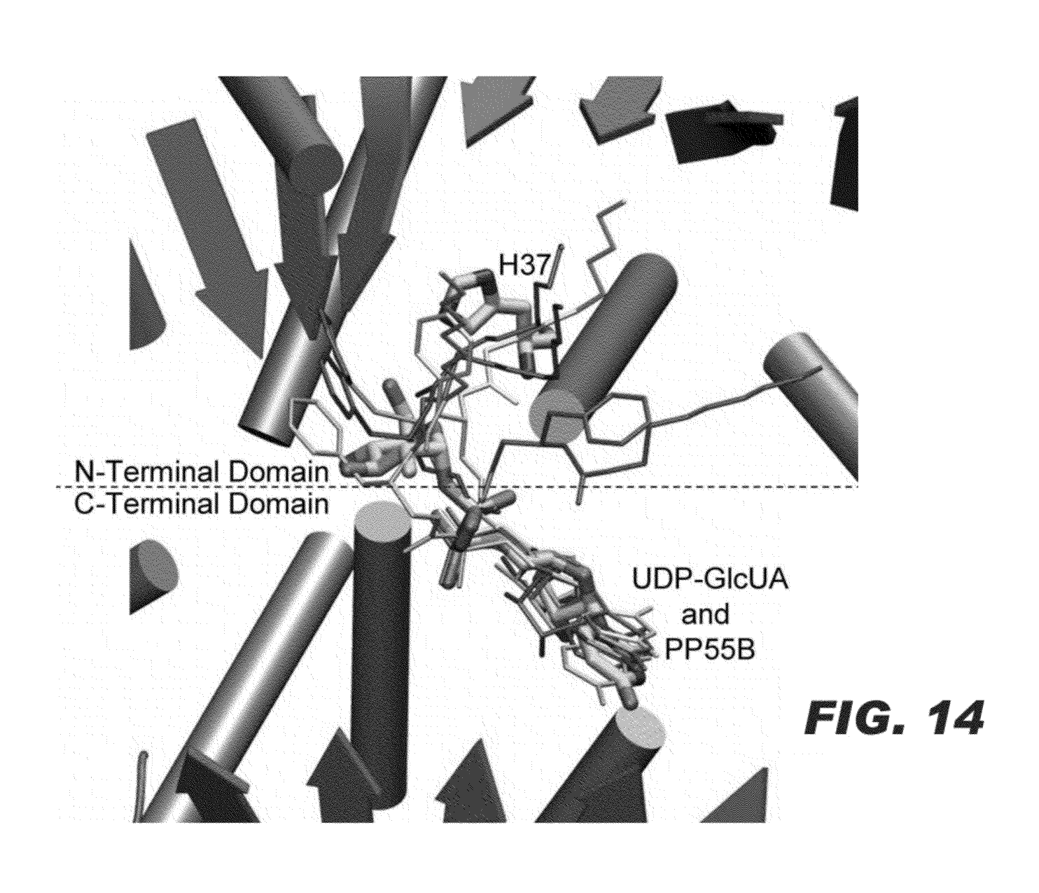

FIG. 14 depicts PP55B in the active site of human UGT1A10 between the N- and C-terminal domains. Five different configurations of PP55B are docked to the active center guided by the sugar moiety.

### DETAILED DESCRIPTION

The present invention encompasses a composition comprising a pharmaceutically active agent and a specific inhibitor of a UGT that glucuronidates the pharmaceutically active agent. Such inhibitors may also be used to decrease the rate of glucuronidation of a compound in a subject. Advantageously, this may decrease the rate of clearance of the compound, and therefore, extend the half-life of the compound. This is particularly advantageous with pharmaceutically active compounds that are rapidly glucuronidated, such as AZT.

### I. Composition of the Invention

One aspect of the present invention encompasses a composition comprising a UGT inhibitor. A UGT inhibitor of the invention binds to the UDP-glucuronic acid binding site of the UGT. As a result, an inhibitor of the invention may inhibit many different UGTs, as each UGT has a UDP-glucuronic acid binding site. In exemplary embodiments, however, a UGT inhibitor of the invention is bidentate. As used herein, “bidentate” refers to the fact that an inhibitor of the invention binds to both the UDP-glucuronic acid binding site and to the substrate binding site. By way of a non-limiting example, a UGT inhibitor of the invention may bind to the UDP-glucuronic acid binding site of UGT2B7 and to the AZT binding site of UGT2B7. As a result, in some embodiments, bidentate inhibitors of the invention are specific for a particular UGT/substrate combination.

Typically, the UGT inhibitor is specific for one or more UGT enzymes, meaning that the inhibitor preferentially inhibits one or more UGT enzymes, as opposed to other, non-UGT enzymes. In some embodiments, the UGT inhibitor may be specific for a single UGT enzyme. In other embodiments, the UGT inhibitor may inhibit more than one UGT enzyme. In still other embodiments, the UGT inhibitor may inhibit the combination of a specific UGT enzyme and substrate.

In certain embodiments, the IC50 of the inhibitor for either UDP-glucuronic acid or substrate is between about 1 μM and about 1000 μM. For instance, the IC50 may be about 1, 5, 10, 15, 20, 25, 30, 35, 40, 45, 50, 55, 60, 65, 70, 75, 80, 85, 90, 95 or 100 μM. Alternatively, the IC50 may be about 100, 200, 300, 400, 500, 600, 700, 800, 900, or 1000 μM. In another embodiment, the IC50 may be less than about 50 μM.

Generally speaking, a UGT inhibitor of the invention may inhibit a UGT from the 1A family. Alternatively, a UGT inhibitor of the invention may inhibit a UGT from the 2B family. For instance, by way of non-limiting example, a UGT inhibitor may inhibit UGT1A6, UGT1A8, UGT1A10, or UGT2B7.

In particular embodiments, the UGT inhibitor may be specific for the combination of a certain UGT and a particular substrate. For instance, a UGT inhibitor may be specific for an exogenous substrate, such as a pharmaceutically active substance, and a UGT that glucuronidates the substrate. Alternatively, a UGT inhibitor may be specific for an endogenous substrate (such as estrogens, reinoids, fatty acids, prostaglandins, bile acids, and various steroid hormones) and a UGT that glucuronidates the endogenous substrate. In some embodiments, a UGT inhibitor is specific for a substrate that is subject to O-linked glucuronidation. In exemplary embodiments, a UGT inhibitor of the invention may be specific for a substrate that is subject to N-linked glucuronidation. Methods of determining if a substrate is subject to N-linked or O-linked glucuronidation are known in the art. In one embodiment, a UGT inhibitor may be specific for AZT and UGT2B7.

In a preferred embodiment, a UGT inhibitor of the invention comprises an N-acyl phenylaminoalcohol residue and a uridine moiety connected by a spacer. These inhibitors are referred to as PP inhibitors. Typically, the substrate specificity of the inhibitor is derived from the N-acyl phenylaminoalcohol residue. Generally speaking, the spacer moiety is designed to mimic the charge and size of the UDP-glucuronic acid phosphate groups that connect the glucuronic acid group to the ribose group. The spacer, while mimicking these groups, does not allow the same UGT catalytic activity as native UDP-glucuronic acid. Typically, the linker may comprise a hydrocarbyl or a substituted hydrocarbyl. For instance, a UGT inhibitor of the invention may comprise a compound illustrated in FIG. 1. In some embodiments, a UGT inhibitor of the invention may be selected from the group consisting of PP36, PP37, PP37P, PP50, PP55(A,B) and PP56B, as illustrated in FIG. 1.

### (a) Pharmaceutically Active Agent

An exemplary aspect of the invention encompasses a composition comprising a pharmaceutically active agent and a specific inhibitor of a UGT that glucuronidates the pharmaceutically active agent. Generally speaking the pharmaceutically active agent is glucuronidated in vivo. In some embodiments, the pharmaceutically active agent is glucuronidated by a single UGT enzyme. In other embodiments, the pharmaceutically active agent is glucuronidated by more than one UGT enzyme. In exemplary embodiments, the pharmaceutically active agent is subject to N-linked glucuronidation. Pharmaceutically active agents that are glucuronidated in vivo are known in the art. Additionally, methods of determining if a pharmaceutically active agent is glucuronidated in vivo are known in the art. For instance, see the method detailed in Miller G P, et al Assessing cytochrome P450 and UDP-glucuronosyltransferase contributions to warfarin metabolism in humans, Chem Res Toxicol. 2009 July; 22(7):1239-45. Suitable, non-limiting examples of pharmaceutically active agents that are glucuronidated in vivo may include AZT, morphine, warfarin, or isolated endogenous estrogens, androgens, reinoids, fatty acids, prostaglandins, bile acids, and various steroid hormones. In one embodiment, the pharmaceutically active agent is AZT. In another embodiment, the pharmaceutically active agent is morphine. In yet another embodiment, the pharmaceutically active agent is warfarin.

### (b) Pharmaceutical Composition

A composition of the invention may be incorporated into a pharmaceutical composition suitable for administration to a subject. A pharmaceutical composition may comprise a UGT inhibitor and a pharmaceutically acceptable carrier. In some embodiments, a pharmaceutical composition may further comprise one or more additional compounds. For instance, a pharmaceutical composition may comprise one or more exogenous compounds or endogenous compounds. Suitable compounds may be glucuronidated in vivo, and may be exogenous or endogenous substrates detailed in Section I above.

As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers may include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the composition.

The pharmaceutical compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. The preferred mode of administration may be parenteral (e.g., intravenous, subcutaneous, intraperitoneal, intramuscular) or oral.

Pharmaceutical compositions may be sterile and are typically stable under the conditions of manufacture and storage. The composition may be formulated as a solution, microemulsion, dispersion, liposome, nanoparticle, or other ordered structure suitable to high drug concentration. Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those detailed above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution may be maintained, for example, by the use of a coating such as lecithin, by the maintenance of the required particle size in the case of dispersion and by the use of surfactants. Prolonged absorption of injectable compositions may be achieved by including an agent that delays absorption, for example, monostearate salts and gelatin, in the composition.

A pharmaceutical composition of the invention may be administered to a subject by a variety of methods known in the art. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, nanoparticules, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody of the invention may be orally administered, for example, with an inert diluent or an assimilable edible carrier. The composition (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation and/or rapid excretion.

Generally speaking, the dosage of inhibitor in a pharmaceutical composition can and will vary based on the inhibitor and the target substrate. Methods of determining dosages are known in the art. In some embodiments, a pharmaceutical composition of the invention may comprise a dosage of between about 0.1 and about 100 mg of a UGT inhibitor. For instance, the composition may comprise about 0.1, 0.5, 1, 5, 10, 15, 20, 25, 30, 35, 40, 45, 50, 55, 60, 65, 70, 75, 80, 85, 90, 95 or 100 mg of inhibitor. In other embodiments, the dosage of the inhibitor will depend directly on the dosage of the substrate. For instance, the ratio of inhibitor to substrate may be between about 0.5:1 to about 1.5:1, including about 0.5:1, 0.6:1, 0.7:1, 0.8:1, 0.9:1, 1:1, 1.1:1, 1.2:1, 1.3:1, 1.4:1, and 1.5:1. In one embodiment where the substrate is AZT, the ratio may be between about 0.9:1 to about 1.1:1.

### II. Methods

Another aspect of the present invention encompasses methods of using a UGT inhibitor. For instance, the present invention provides a method for decreasing the rate of glucuronidation of a compound in a subject. Decreasing the rate of glucuronidation may decrease the rate of clearance and/or increase the half-life of the compound. The method typically comprises administering to the subject both the compound and a UGT inhibitor specific for a UGT that glucuronidates the compound at substantially the same time.

As used herein, “at substantially the same time” means within about 48, 46, 44, 42, 40, 38, 36, 34, 32, 30, 28, 26, 24, 22, 20, 18, 16, 14, 12, 10, 8, 6, 4, 2, or 1 hours. In one embodiment, at substantially the same time means within about 60, 50, 40, 35, 30, 25, 20, 15, 10, 5, 4, 3, 2, or 1 minutes. In some embodiments, the compound is administered first. In other embodiments, the inhibitor is administered first. In an alternative embodiment, the compound and the inhibitor are administered simultaneously.

The compound and the inhibitor may be administered in the same way, or alternatively, in different ways. By way of non-limiting example, the compound and the inhibitor may both be administered orally, or one may be administered intravenously and the other orally. Suitable routes of administration are detailed in Section I above.

The compound may be an exogenous compound or an endogenous compound. Suitable exogenous compounds may include, for instance, pharmaceutically active compounds and exogenous UGT substrates and are detailed in section I above. Suitable endogenous compounds may include endogenous UGT substrates, which are also detailed in section I above.

In one embodiment, the invention encompasses a method for decreasing the clearance of AZT in a subject. The method comprises administering an inhibitor specific for UGT2B7 at substantially the same time as the AZT. In another embodiment, the invention encompasses a method for increasing the half-life of AZT in a subject. The method comprises administering an inhibitor specific for UGT2B7 at substantially the same time as the AZT.

In another embodiment, the invention encompasses a method for decreasing the clearance of Midazolam in a subject. The method comprises administering an inhibitor specific for UGT1A4 at substantially the same time as the Midazolam. In another embodiment, the invention encompasses a method for increasing the half-life of Midazolam in a subject. The method comprises administering an inhibitor specific for UGT1A4 at substantially the same time as the Midazolam.

In yet another embodiment, the invention encompasses a method for decreasing the clearance of morphine in a subject. The method comprises administering an inhibitor specific for UGT2B7 at substantially the same time as the morphine. In another embodiment, the invention encompasses a method for increasing the half-life of morphine in a subject. The method comprises administering an inhibitor specific for UGT2B7 at substantially the same time as the morphine.

### DEFINITIONS

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, carbocycle, aryl, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention. Therefore all matter set forth or shown in the accompanying drawings is to be interpreted as illustrative and not in a limiting sense.

### EXAMPLES

The following examples illustrate various iterations of the invention.

### Example 1

### Introduction

Glucuronidation of endo- and xenobiotic compounds takes place in the endoplasmic reticulum by transfer of glucuronic acid from UDP-glucuronic acid (UDP-GlcUA) to an acceptor group and is carried out by membrane-bound UDP-glucuronosyltransferases (EC 2.4.1.17, UGT). Although glucuronidation reactions and UGT properties have been investigated extensively for many years, studies have only just begun to give details of UGT structure. Recently, we presented the first 1.8 angstrom resolution apo-crystal structure of the C-terminal UDP-GlcUA binding domain of human UGT2B7 (1). This enabled us to create the first reliable models of this region and predict important contact amino acids involved in co-substrate binding. This information is of great importance due to the similarities among the C-terminal domains of all human UGTs.

The aim of this work is to characterize the active site and the catalytic mechanism of UGT2B7 by focusing on the interactions between the co-substrate and substrate binding sites of human UGTs, using 8 specific bidentate inhibitors. Inhibitors are a valuable tool for the study of the active site structure and its relation to enzyme function. The “PP” inhibitors used here are composed of N-acyl phenylaminoalcohol derivatives linked to uridine via different spacers (FIG. 1). These compounds were developed as inhibitors of membrane-bound glycosyltransferases and several have been found to be competitive inhibitors of glucosyl phosphoryldolichol synthase activity (2). Previous studies have also been published that analyze their action as inhibitors of rat and human liver microsomal UGTs (3, 4) and human recombinant UGT1A6 (5). Here, we have investigated the inhibitory potency of this series of uridinyl analog inhibitors on glucuronidation by UGT2B7 and compared it to that seen with UGT1A10.

### Materials and Methods

Recombinant UGT Isoform Incubations—Recombinant human UGT2B7 was produced in HEK239 cells as previously described (6). UGT1A10 was produced as a His-tagged protein in baculovirus-infected Sf9 insect cells as previously described (7, 8). Synthesis of inhibitors was described previously (2). For inhibition assays, recombinant UGT membrane protein was incubated in reaction buffer with substrate, inhibitor, and co-substrate as previously described (6, 9).

Screening assays—UGT1A6, 1A8, 1A10 and 2B7 were incubated with fixed concentrations of each substrate and co-substrate and two concentrations of each inhibitor (FIG. 2).

IC₅₀ assays—UGT2B7 or 1A10 were incubated with fixed concentrations of each substrate and co-substrate and increasing concentrations of inhibitor. To determine IC_{50(substrate)} a concentration of substrate equal to ˜2 times Km was used while co-substrate was added at a >5 fold molar excess. To determine IC_{50(UDP-GlcUA)}, a concentration of co-substrate equal to ˜2 times Km was used while substrate was added at a >5 fold excess (FIG. 3).

Ki assays—UGT2B7 or 1A10 were incubated with a range of at least four different concentrations of inhibitor using varying concentrations of substrate (Ki_{(substrate)}) with a fixed saturating concentration of cosubstrate or with varying concentrations of co-substrate (Ki_{(UDP-GlcUA)}) and a fixed saturating concentration of substrate (FIG. 4).

The results of these experiments were analyzed and apparent kinetic and inhibition parameters were determined using Prism 4 software (GraphPad, San Diego, Calif.) and DynaFit (BioKin, Watertown, Mass.). TLC and HPLC Analysis—For inhibition assays using all substrates other than 7-OH-warfarin, aliquots (60 μl) of each incubation were analyzed using thin layer chromatography (TLC) as previously described (3). For inhibition assays using 7-OH-warfarin as a substrate, samples were analyzed using HPLC as described previously (10). Primary standards for the 7-OH-warfarin glucuronide were not available; therefore, product concentrations were estimated using the external standard response for the 7-OH-warfarin substrate. It has been shown previously that the addition of the glucuronic acid moiety does not alter the extinction coefficient from that of the unreacted substrate (5).

Homology modeling of human UGT1A10—A model of the UDP-GlcUA binding site of UGT1A10 was built using the structure of this domain in UGT2B7 as a template. The model structure (FIG. 5), produced in PyMOL (http://www.pymol.org), shows the bound co-substrate and the location of key residues in either UGT1A10 or UGT2B7 within hydrogen bonding distance. The residue types and numbering are indicated for both UGT1A10 (black) and UGT2B7 (green). A model of the mature UGT2B7 was built with the program Modeller, version 9v4 (9). The template proteins (2o6l, 2vce and 2iyf) were aligned structurally and the UGT sequences were added stepwise to the structural alignment with the program ClustalW v2.0.5 (10). The Cterminal helix missing from the templates was built de novo. The uridinyl-sugar moieties of the templates were transferred to the UGT protein at construction. The model was optimimized within Modeller with CHARMM forcefield. Structures of the inhibitors were optimized with the semi-empirical quantum chemistry program MOPAC2007, version 8.148L (JJP Stewart, Fujitsu Limited, Tokyo). The substrate and inhibitor structures were docked manually to the protein model, guided by the substrate structure (FIGS. 6-9).

### Results

Inhibitors were screened for their ability to inhibit UGT1A6, -1A8, -1A10 activity towards 4-MU, a substrate common to these isoforms. UGT2B7 inhibition was measured with linoleic acid, since 4-MU is not a substrate for this isoform. All inhibitors were shown to affect all four UGTs with PP36 and 37P having the least effect and PP50(A/B) and 55(A/B) having the strongest inhibitory effects (FIG. 2). UGT1A8 was very sensitive to the inhibitors, showing high levels of inhibition at low inhibitor concentrations. UGT2B7 was seemingly more robust and required higher concentrations of inhibitor to cause significant decreases in activity for all inhibitors other than PP55A and B (this could be due in part to the different substrate used for analysis). PP55B was chosen for more detailed analysis with UGT2B7 and comparison to UGT1A10.

In order to determine the inhibitory potency of PP55B on the enzymatic activity of UGT2B7 toward androsterone, linoleic acid, and pNP, IC₅₀ values for substrate inhibition were determined (FIG. 3). IC₅₀ values were lowest for androsterone inhibition and ranged from 3.3 μM for PP55B to 210 μM for PP36. IC₅₀ values for linoleic acid were also relatively low and ranged from 14 μM for PP55B to 740 μM for PP36. IC₅₀ values for pNP, not a natural substrate, varied greatly and were difficult to measure accurately in our system. These values ranged from 45 μM for PP55B to greater than 1 mM for PP36 and PP56B.

In order to further characterize the inhibition of UGT2B7 activity by PP55B, apparent Ki_{(substrate)} and Ki_{(UDP-GlcUA)} values were calculated using DynaFit (FIG. 4, Table 1). Ki_{(substrate)} values for UGT2B7 activity toward all substrates were low (Androsterone: 1.1 μM; Linoleic Acid: 4.2 μM; pNP: 7.6 μM). Ki_{(UDP-GlcUA)} values for UGT2B7 activity toward all substrates were low and almost identical (Androsterone: 2.2 μM; Linoleic Acid: 2.5 μM; pNP: 2.1 μM).

TABLE 1
Km, IC50, and Ki values as well as inhibition type for the inhibition of UGT2B7
and -1A10 glucuronidation activity towards selected substrates by PP55B.

| | | Substrate | | | | | UDP-GlcUA | | | | |
|---|---|---|---|---|---|---|---|---|---|---|---|
| | | Km | IC50 | Kiapp | Ki′app | Inhibition | Km | IC50 | Kiapp | Ki′app | Inhibition |
| Isoform | Substrate | (μM) | (μM) | (μM) | (μM) | Type | (μM) | (μM) | (μM) | (μM) | Type |
| UGT2B7 | Androsterone | 22 ± 2 | 3.7 ± 0.3 | 1.0 | 4.7 | Mixed | 520 ± 60 | — | 2.2 | 59 | Mixed |
| | Linoleic Acid | 160 ± 20 | 14 ± 0.6 | 4.2 | | Competitive | 1500 ± 100 | — | 2.5 | | Competitive |
| | p-Nitrophenol | 760 ± 130 | 75 ± 10 | 7.6 | | Competitive | 740 ± 90 | — | 2.1 | 85 | Mixed |
| UGT1A10 | 7-OH Warfarin | 450 ± 40 | 11 ± 2 | 8.5 | | Competitive | 970 ± 70 | 4.9 ± 0.2 | 4.1 | | Competitive |
| | 8-OH Warfarin | 510 ± 70 | 35 ± 4 | 27 | 136 | Mixed | 710 ± 80 | 14 ± 1 | 26 | | Competitive |
| | 4-MU | 200 ± 20 | 8 ± 1 | 96 | | Non- | 240 ± 60 | 15 ± 2 | 5.7 | | Competitive |
| | | | | | | competitive | | | | | |
| | E₂ | 46 ± 5 | ~200* | 200 | 43 | Mixed | 510 ± 100 | 39 ± 11 | 6.9 | 23 | Mixed |

“—” indicates not calculated.
The inhibition constant Kiapp refers to the dissociation for the enzyme-inhibitor combination (E + I [symbol] EI). The inhibition constant Ki′app refers to the dissociation for the enzyme/substrate-inhibitor combination (EA + I [symbol] EAI).
*Some activation is seen at low concentrations so calculation value is an estimation. UGT1A10.

The type of inhibition was also determined for each reaction using DynaFit (FIG. 4, Table 1). Androsterone showed mixed inhibition of both substrate and co-substrate binding. Linoleic acid showed competitive inhibition of both substrate and co-substrate binding. pNP showed competitive inhibition of substrate binding and mixed inhibition of co-substrate binding.

To shed new light on the apparent differences between UGT1A10 and UGT2B7 binding, a model of UDP-GlcUA bound to UGT1A10 and UGT2B7 was built (FIG. 5). The structure of the UDP-GlcUA binding fragment of UGT2B7 was used as a template to generate a model of the C-terminal domain of UGT1A10 (11). UDP-GlcUA was manually docked into the UGT2B7/1A10 partial model in the same conformation as UDP-glucose in VvGT1 (12). The docking shows that H369, E378, D393 and Q394 (UGT1A10 numbering) are arranged around the UDP-GlcUA. S372 and H373 (UGT1A10 numbering) appear to be important, based on the docked model. These two residues can hydrogen bond to the UDP-GlcUA. The changes at these residues may thus explain some of the differences in UDP-GlcUA binding between UGT1A10 and UGT2B7, as S372 is replaced by Ala, and H373 by Asn in UGT2B7, respectively.

A homology model of UGT2B7 was built, based on information from its crystal structure and sequence homology to available structures of plant glucosyltransferases with the GT-B folding motif. The model highlights the proposed location of His37, which putatively participates in the activation of the aglycon substrate (1). It also shows the binding site of the sugar donor, UDP-glucuronic acid (UDPGA), and the substrates pNP and androsterone in close proximity to His37 (FIG. 6).

We have used this model to dock the inhibitor PP55B at the UDPGA binding site through the uridine group of the inhibitor. A possible orientation of the inhibitor, when bound to the UDP-GlcUA binding site, with respect to the N-terminal domain and, particularly, His37, is shown (FIGS. 7, 8, and 9).

### Conclusion

The aim of this work was to characterize the active site and the catalytic mechanism of UGT2B7 using active site directed bidentate inhibitors and homology modeling, and to compare this information to that generated for UGT1A10. We can predict the binding mode for the inhibitor based on the inhibition mechanism.

Three types of inhibition are seen in the data for UGT2B7 and UGT11A10. Competitive inhibition indicates that inhibition is caused by PP55B binding to the site for the molecule being varied but NOT by binding to the other site. Mixed inhibition posits that inhibition is caused by PP55B binding to both substrate and cofactor sites during turnover, individually and/or simultaneously. Noncompetitive inhibition indicates that inhibition is cause by PP55B binding to the site of the molecule that is NOT being varied, and thus titrates out active enzyme.

For these catalytic studies, substrate or co-substrate is present in excess. PP55B binding then reflects the association of the inhibitor with a binary enzyme complex. When the concentration of the co substrate is in excess, inhibition of linoleic acid and pNP glucuronidation activity in UGT2B7 and 7-OH-warfarin glucuronidation activity in UGT1A10 is competitive and caused by PP55B binding to the substrate binding site. Inhibition of androsterone glucuronidation activity in UGT2B7 and 8-OH-warfarin and E2 glucuronidation activity in UGT1A10 is mixed and caused by PP55B binding to both the substrate and co-substrate sites, individually and/or simultaneously. Inhibition of 4-MU glucuronidation activity in UGT1A10 is noncompetitive and may indicate that binding of 4-MU occurs after the association of PP55B to the cofactor binding site.

When the concentration of the substrate is in excess, inhibition of linoleic acid glucuronidation activity in UGT2B7 and 7-OH, 8-OH-warfarin and 4-MU glucuronidation activity in UGT1A10 is competitive and caused by PP55B binding to the co-substrate binding site. Inhibition of androsterone and pNP glucuronidation activity in UGT2B7 and E₂ glucuronidation activity in UGT1A10 is mixed and caused by PP55B binding to both the substrate and co-substrate sites, individually and/or simultaneously. S372 and H373 in UGT1A10 and the corresponding A337 and N378 in 2B7 appear to be important, based on the docked model. These two residues can hydrogen bond to the UDP-GlcUA and the changes at these residues may thus explain some of the differences in UDP-GlcUA binding between UGT1A10 and UGT2B7.

Docking of PP55B into the active site of UGT2B7 shows that this inhibitor acts as a transition state analog to inhibit glucuronidation activity. It can simultaneously bind in both the substrate and co-substrate sites. Overall, the PP inhibitors interact with the co-substrate binding site of both UGT1A10 and 2B7 in a similar manner as reflected by the similar Ki_{(cosubstrate)} values. What makes these inhibitors isoform/substrate-selective is their interactions with the different substrate binding motifs found in each isoform. Combined interactions within both the substrate and co-substrate binding sites can be explored further to achieve maximum isoform/substrate specific inhibition.

### References for Example 1

1. Miley, M J, Zielinska, A K, Keenan, J E, Bratton, S M, Radominska-Pandya, A, and Redinbo, M R. J Mol Biol, 2007. 369(2): p. 498-511.
2. Paul, P, Lutz, T M, Osborn, C, Kyosseva, S, Elbein, A D, Towbin, H, Radominska, A, and Drake, R R. J Biol Chem, 1993. 268(17): p. 12933-8.
3. Little, J M, Drake, R R, Vonk, R, Kuipers, F, Lester, R, and Radominska, A. J Pharmacol Exp Ther, 1995. 273(3): p. 1551-9.
4. Radominska, A, Berg, C, Treat, S, Little, J M, Lester, R, Gollan, J L, and Drake, R R. Biochim Biophys Acta, 1994. 1195(1): p. 63-70.
5. Battaglia, E, Elass, A, Drake, R R, Paul, P, Treat, S, Magdalou, J, Fournel-Gigleux, S, Siest, G, Vergoten, G, Lester, R, and et al. Biochim Biophys Acta, 1995. 1243(1): p. 9-14.
6. Gall, W E, Zawada, G, Mojarrabi, B, Tephly, T R, Green, M D, Coffman, B L, Mackenzie, P I, and Radominska-Pandya, A. J Steroid Biochem Mol Biol, 1999. 70(1-3): p. 101-8.
7. Kurkela, M, Garcia-Horsman, J A, Luukkanen, L, Morsky, S, Taskinen, J, Baumann, M, Kostiainen, R, Hirvonen, J, and Finel, M. J Biol Chem, 2003. 278(6): p. 3536-44.
8. Kuuranne, T, Kurkela, M, Thevis, M, Schanzer, W, Finel, M, and Kostiainen, R. Drug Metab Dispos, 2003. 31(9): p. 1117-24.
9. Little, J M, Kurkela, M, Sonka, J, Jantti, S, Ketola, R, Bratton, S, Finel, M, and Radominska-Pandya, A. J Lipid Res, 2004. 45(9): p. 1694-703.
10. Zielinska, A, Lichti, C F, Bratton, S, Mitchell, N C, Gallus-Zawada, A, Le, V H, Finel, M, Miller, G P, Radominska-Pandya, A, and Moran, J R J Pharmacol Exp Ther, 2008. 324(1): p. 139-48.
11. Miley, M, Zielinska, A, Keenan, J, Bratton, S, Radominska-Pandya, A, and Redinbo, M. J Mol Biol, 2007: p. IN PRESS.
12. Offen, W, Martinez-Fleites, C, Yang, M, Kiat-Lim, E, Davis, B G, Tarling, C A, Ford, C M, Bowles, D J, and Davies, G J. Embo J, 2006. 25(6): p. 1396-405.

### Example 2

### Introduction

The glucuronidation of endo- and xenobiotic compounds takes place in the endoplasmic reticulum (ER) by transfer of glucuronic acid from UDP-glucuronic acid (UDP-GlcUA) to an acceptor group, and is carried out by membrane-bound UDP-glucuronosyltransferases (EC 2 4 1 17 UGT) Although glucuronidation reactions and UGT properties have been extensively investigated (1), several important aspects of UGTs, including the structure of the active site, have not been elucidated. Specific inhibitors are a valuable tool for the study of the active site structure and its relation to enzyme function.

Several compounds have been shown to specifically inhibit UGTs in vitro. One class of compounds, N-acyl phenylaminoalcohol derivatives, linked to uridine via different spacers (structures are shown in FIG. 1), were developed as inhibitors of membrane-bound glycosyltransferases, several of which were found to be competitive inhibitors of glucosyl phosphoryldolichol synthase activity and photolabeling (2). Subsequently, these uridinyl analogs were evaluated as inhibitors of rat and human liver microsomal UGT activity and photolabeling (3, 4) and human recombinant UGT1A6 activity (5). With human recombinant UGT1A6, PP37 (DHPASU), PP37P (DHPASiU), PP5OB (d-DPASiU) and PP56B (d-DPMSU) were identified as inhibitors of 4-methylumbelliferone (4-MU) glucuronidation. In all cases, the inhibition was competitive toward UDP-GlcUA, however, with 4-MU, the inhibition was non-competitive for PP50B and competitive for the other three compounds.

Here the potency of the inhibitors on UGT1A10 mediated glucuronidation was investigated for several different substrates: 4-MU, E₂, 7-OH and 8-OH warfarin. IC₅₀s have been determined for all the available inhibitors and Ki values for both substrate and UDP-GlcUA have been evaluated with the most effective inhibitor, PP55B.

### Materials and Methods

Recombinant UGT Isoform Incubations—Recombinant human UGT1A10 and it mutants were produced in baculovirus-infected insect cells as previously described (6, 7). Synthesis of inhibitors was described previously (2). For inhibition assays, UGT recombinant membrane protein (5 μg) was incubated in reaction buffer (100 μM Tris-HCl (pH 7.4)/5 mM MgCl₂/5 mM saccharolactone) and 2% DMSO with substrate, inhibitor and co-substrate, UDP-GlcUA, in a total volume of 30 μl. Substrates were added in DMSO, and controls were run with each assay. No additional detergents or other activators were used in the incubations. Reactions were started by the addition of co-substrate and incubated at 37° C. (90 min for 8-hydroxywarfarin; 30 min for 4-MU; 30 min for E₂). The rate of glucuronidation of 8-hydroxywarfarin was shown to be linear for up to 3 h. Reactions were stopped by addition of 40 μl of ethanol, and all incubations were performed in duplicate. The results of these experiments were analyzed and apparent kinetic and inhibition parameters were determined using Prism 4 software (GraphPad, San Diego, Calif.) and DynaFit (BioKin, Watertown, Mass.).

IC₅₀ assays—UGT1A10 was incubated with a fixed concentration of each substrate (4-MU: 250 μM; 7- and 8-hydroxywarfarin: 500 μM; E₂: 25 μM) and co-substrate (2 mM) and increasing concentrations of inhibitor (0-500 μM).

Ki assays—UGT1A10 was incubated with at least four different concentrations of inhibitor using varying concentrations of Substrate (Ki_{(substrate)}) with a fixed saturating concentration of co-substrate or with varying concentrations of co-substrate (Ki_{(UDP-GlcUA)}) with a fixed saturating concentration of substrate (4-MU, 7- and 8-hydroxywarfarin: 3 mM; E₂: 1 mM).

TLC Analysis—For inhibition assays using 4-MU, E₂, and 8-OH warfarin as substrates, aliquots (60 μl) of each sample were analyzed using thin layer chromatography (TLC) as described previously (3).

HPLC Analysis—For inhibition assays using 7-OH warfarin as a substrate, samples were analyzed using HPLC. Each sample was centrifuged at 14,000 rpm for 8 min to spin down the protein, and 60 μl of the supernatant was analyzed by HPLC as described previously (8). Primary standards for the 7-OH warfarin glucuronide were not available; therefore, product concentrations were calculated using the external standard response for each warfarin substrate. It has been shown previously that the addition of the glucuronic acid moiety does not alter the extinction coefficients from that of the unreacted substrate (5).

Homology modeling of human UGT1A10—A model of the mature UGT1A10, residues 25-529, was built with the program Modeller, version 9v4 (9). The template proteins (2061, 2vce and 2iyf) were aligned structurally, and the UGT sequences were added stepwise to the structural alignment with the program ClustalW v2.0.5 (10). The C-terminal helix missing from the templates was built de novo. The uridinyl-sugar moieties of the templates were transferred to the UGT protein at construction. The model was optimimized within Modeller with CHARMM forcefield. Structures of the inhibitors were optimized with the semi-empirical quantum chemistry program MOPAC2007, version 8.148L (JJP Stewart, Fujitsu Limited, Tokyo). The inhibitor structures were docked manually to the protein model, guided by the substrate structure.

### Results

Inhibitors were screened for their ability to inhibit UGT1A6, -1A8, and 1A10 activity towards 4-MU, a substrate common for these isoforms. All inhibitors were shown to affect all three UGTs with PP37P having the least effect and PP50(A/B)-55(A/B) having the strongest inhibitory effects (FIG. 10).

Screening led to the selection of UGT1A10 and PP55B for further analysis. In order to determine the inhibitory potency of PP55B on the enzymatic activity of UGT1A10 toward 4-MU, 7- and 8-hydroxywarfarin, and E₂, IC₅₀ values for both substrate and co-substrate inhibition were determined (FIG. 11, Table 2). IC_{50 (substrate)} values for the 3 drugs were similar and ranged from approximately 8 to 33 μM. IC₅₀ (_{substrate}) values for E₂ were not calculated due to apparent activation of glucuronidation at lower inhibitor concentrations. IC_{50 (UDP-GlcUA)} values determined with all substrates were very low ranging from about 5 to 40 μM. PP55B did not fully inhibition UGT1A10 activity toward 7-OH warfarin.

TABLE 2
Km, IC50, and Ki values as well as inhibition type for the inhibition of
UGT1A10 glucuronidation activity towards selected substrates by PP55B.

| | Substrate | | | | UDP-GlcUA | | | |
|---|---|---|---|---|---|---|---|---|
| | Km | IC50 | Kiapp | Inhibition | Km | IC50 | Kiapp | Inhibition |
| Substrate | (μM) | (μM) | (μM) | Type | (μM) | (μM) | (μM) | Type |
| 7-OH | 497 ± 61 | 11.3 | 8.7 | Competitive | 985 ± 85 | 4.9 | — | Mixed |
| Warfarin | | 2.2 | | | | 0.2 | | |
| 8-OH | 515 ± 17 | 33.6 | 16 | Competitive | 785 ± 127 | 13.9 | 26 | Competitive |
| Warfarin | | 3.7 | | | | 1.1 | | |
| 4-MU | 91.1 ± 19 | 8.0 | 16 | Competitive | 245 ± 85 | 14.8 | 5.5 | Competitive |
| | | 1.0 | | | | 1.8 | | |
| E₂ | 48.3 ± 9 | — | 42 | Uncompetitive | 116 ± 36 | 39.0 | — | Mixed |
| | | | | | | 10.8 | | |

“—” indicates not calculated.

In order to further characterize the inhibition of UGT1A10 activity by PP55B, apparent Ki_{(substrate)} and Ki_{(UDP-GlcUA)} values were determined (FIG. 12, Table 2). Ki_{(substrate)} values were similar ranging from 5 to 17 μM. The apparent Ki_{(substrate)} value could not be calculated for E₂ because the IC₅₀ value was not calculated. Ki_{(UDP-GlcUA)} were very similar differing by less than 4 μM.

The type of inhibition was also determined for each reaction (FIG. 12, Table 2). 8-hydroxywarfarin and 4-MU showed competitive inhibition of both substrate and co-substrate binding. 7-hydroxywarfarin showed competitive inhibition of substrate binding but mixed co-substrate inhibition. E₂ glucuronidation showed mixed inhibition of co-substrate binding by PP55B and uncompetitive inhibition of substrate binding.

A new model of UGT1A10 was built, based on sequence homology to available structures of plant glucosyltransferases with the GT-B folding motif. The model highlights the proposed location of His37 that probably participate in the activation of the aglycon substrate (11) and the conserved aspartic acid residue, Asp148, that may stabilize His37.The model also shows the binding site of the sugar donor, UDP-glucuronic acid (UDPGA), at the C-terminal domain of the enzyme, but in close proximity to His37 within the N-terminal domain (FIG. 13).

We have used this model to dock the inhibitor PP55B at the UDPGA binding site through the uridine group of the inhibitor. A possible orientation of the inhibitor, when bound to the UDPGA binding site, with respect to the N-terminal domain and, particularly, His37, is shown in FIG. 14.

### Conclusion

The aim of this work was to characterize the active site and the catalytic mechanism of UGT1A10 using active site directed inhibitors and homology modeling. As bidentate inhibitors, i.e. bind to both sites, we can predict the binding mode for the inhibitor based on the inhibition mechanism. Competitive inhibition indicates that PP55B binds to the site for the molecule binding varied but NOT the other site. In the case for 4-MU and 8-hydroxywarfarin the inhibitor only binds to a SINGLE site in the presence of substrate and inhibitor. Uncompetitive inhibition indicates PP55B binds to the site of the molecule that is NOT being varied, and thus titrates out active enzyme. This mechanism may apply to E₂ turnover indicating that binding of E₂ favors the association of PP55B to the cofactor binding site. Mixed inhibition posits that PP55B is binding to both substrate and cofactor sites during turnover. Whether this process can occur simultaneously is unclear at this point.

For these catalytic studies, substrate or co-substrate is present in excess. PP55B binding then reflects the association of the inhibitor with a binary enzyme complex. The presence of different substrates at the active site could alter the cofactor binding site and consequently the “cofactor-like” region of the inhibitor. The Ki values with respect to the cofactor site could then vary among the particular substrate reactions. By contrast, bound cofactor would yield the same UGT complex and corresponding active site for substrate, and thus the “substrate-like” portion of the inhibitor should competitively bind to this site with the same Ki value in all catalytic studies. This seems to be the case for 7-hydroxywarfarin (Ki 8.7 μM), 8-hydroxywarfarin (Ki 16 μM), and 4-MU (Ki 16 μM), but not E₂ (mixed).

If valid, the unusual parameters for E₂ may indicate more complex mechanisms including the possibility of multiple substrate binding sites leading to allosteric effects.

### References for Example 2

1. Radominska-Pandya, A, Czernik, P J, Little, J M, Battaglia, E, and Mackenzie, Pl. Drug Metab Rev, 1999. 31(4): p. 817-99.
2. Paul, P, Lutz, T M, Osborn, C, Kyosseva, S, Elbein, A D, Towbin, H, Radominska, A, and Drake, R R. J Biol Chem, 1993. 268(17): p. 12933-8.
3. Little, J M, Drake, R R, Vonk, R, Kuipers, F, Lester, R, and Radominska, A. J Pharmacol Exp Ther, 1995. 273(3): p. 1551-9.
4. Radominska, A, Berg, C, Treat, S, Little, J M, Lester, R, Gollan, J L, and Drake, R R. Biochim Biophys Acta, 1994. 1195(1): p. 63-70.
5. Battaglia, E, Elass, A, Drake, R R, Paul, P, Treat, S, Magdalou, J, Fournel-Gigleux, S, Siest, G, Vergoten, G, Lester, R, and et al. Biochim Biophys Acta, 1995. 1243(1): p. 9-14.
6. Kurkela, M, Garcia-Horsman, J A, Luukkanen, L, Morsky, S, Taskinen, J, Baumann, M, Kostiainen, R, Hirvonen, J, and Finel, M. J Biol Chem, 2003. 278(6): p. 3536-44.
7. Kuuranne, T, Kurkela, M, Thevis, M, Schanzer, W, Finel, M, and Kostiainen, R. Drug Metab Dispos, 2003. 31(9): p. 1117-24.
8. Zielinska, A, Lichti, C F, Bratton, S, Mitchell, NC, Gallus-Zawada, A, Le, V H, Finel, M, Miller, G P, Radominska-Pandya, A, and Moran, J H. J Pharmacol Exp Ther, 2008. 324(1): p. 139-48.
9. Sali, A and Blundell, T L. J Mol Biol, 1993. 234(3): p. 779-815.
10. 10.Larkin, Mass., Blackshields, G, Brown, N P, Chenna, R, McGettigan, P A, McWilliam, H, Valentin, F, Wallace, I M, Wilm, A, Lopez, R, Thompson, J D, Gibson, T J, and Higgins, D G. Bioinformatics, 2007. 23(21):
11. p.2947-8.
12. 11.Miley, M J, Zielinska, A K, Keenan, J E, Bratton, S M, Radominska-Pandya, A, and Redinbo, M R. J Mol Biol, 2007. 369(2): p. 498-511.

What is claimed is:

1. A composition, the composition comprising a pharmaceutically active agent and a specific inhibitor of a UGT that glucuronidates the pharmaceutically active agent.

2. The composition of claim 1, wherein the composition comprises a specific inhibitor of UGT2B7.

3. The composition of claim 2, wherein the pharmaceutically active agent is AZT.

4. The method of claim 1, wherein the pharmaceutically active agent is Midazolam.

5. The method of claim 1, wherein the pharmaceutically active agent is morphine.

6. The composition of claim 1, wherein the inhibitor is a bidentate inhibitor.

7. The composition of claim 1, wherein the specific inhibitor comprises a N-acyl phenylaminoalcohol residue and a uridine moiety connected by a spacer.

8. The composition of claim 1, wherein the specific inhibitor is selected from the group consisting of PP36, PP37, PP37P, PP50, PP55(A,B) and PP56B.

9. A method for decreasing the rate of glucuronidation of a compound in a subject, the method comprising administering the compound to the subject at substantially the same time as a UGT inhibitor specific for a UGT that glucuronidates the compound is administered.

10. The method of claim 9, wherein the compound is an exogenous compound.

11. The method of claim 9, wherein the compound is an endogenous compound.

12. The method of claim 10, wherein the compound is AZT.

13. The method of claim 10, wherein the compound is Midazolam.

14. The method of claim 10, wherein the compound is morphine.

15. The composition of claim 9, wherein the inhibitor is a bidentate inhibitor.

16. The method of claim 9, wherein the inhibitor comprises a N-acyl phenylaminoalcohol residue and a uridine moiety connected by a spacer.

17. The method of claim 9, wherein the subject is human.

18. The method of claim 12, wherein the UGT inhibitor is UGT2B7.

19. The method of claim 14, wherein the UGT inhibitor is UGT2B7.
